(19) United States
(12) Patent Application Publication
(10) Pub. No.: US 2026/0020754 A1
(43) Pub. Date: Jan. 22, 2026

(54) Illumination device, imaging device having an illumination device, imaging system, method for generating illumination light, and method for operating an imaging device

(71) Applicant: KARL STORZ SE & Co KG, Tuttlingen (DE)

(72) Inventors: Lukas Buschle, Mühlheim (DE); Werner Göbel, Überlingen (DE); Antje Haap-Hoff, Jungingen (DE); Roland Frohberg, Tuttlingen (DE); Klaus Hammerl, Tuttlingen (DE); Florian Huber, Durchhausen (DE)

(73) Assignee: KARL STORZ SE & Co KG, Tuttlingen (DE)

(21) Appl. No.: 18/994,392

(22) PCT Filed: Jul. 13, 2023
(86) PCT No.: PCT/EP2023/069491

§ 371 Date: Jan. 14, 2025

(30) Foreign Application Priority Data
Jul. 14, 2022 (DE) 10 2022 117 578.2
Jul. 14, 2022 (DE) 10 2022 117 581.2
Jul. 27, 2022 (DE) 10 2022 117 580.4

(51) Int. Cl. A61B 1/06; A61B 1/00; A61B 1/04

(52) U.S. Cl. CPC A61B 1/0638; A61B 1/00126; A61B 1/043; A61B 1/0646; A61B 1/0669; A61B 1/0684

(57) ABSTRACT

The invention relates to an illumination device (12), in particular for providing illumination light for an imaging unit (14) such as an endoscope, exoscope and/or microscope, comprising: an optical interface (16) for the optical connection of an imaging unit (14); and an illumination unit (18) that is designed to provide illumination light to the optical interface (16). The invention also relates to an imaging device (10), in particular a medical imaging device, in particular an endoscopic and/or exoscopic and/or microscopic imaging device, comprising: an illumination device (12) having an optical interface (16) and an illumination unit (18) for providing illumination light for an imaging unit (14); an imaging unit (14), which can be connected to the optical interface (16) of the illumination device (12); and a controller (66), which is configured to automatically match an operating state of the imaging unit (14) and an illumination mode of the illumination unit (18) to one another. The invention also relates to associated systems and methods.

The invention relates to an illumination device, to an imaging device, in particular a medical one, having an illumination device, to an imaging system, in particular a medical one, to a method for generating illumination light, and to a method for operating an imaging device, in particular a medical one.

Imaging devices, such as endoscopic or exoscopic devices that produce multispectral or hyperspectral images, are known from the prior art. Multispectral or hyperspectral images have a spectral dimension in addition to two spatial dimensions, such as a conventional image from a camera. The spectral dimension includes multiple spectral bands (wavelength bands). Multispectral and hyperspectral images differ substantially in the number and width of their spectral bands.

Some imaging devices are known for producing such multispectral or hyperspectral images, especially in the context of medical applications. For example, DE 20 2014 010 558 U1 describes a device for acquiring a hyperspectral image of an examination region of a body. The device includes an input lens for generating an image in an image plane and a slit-shaped aperture in the image plane for masking out a slit-shaped region of the image. The light passing through the aperture is spread out by a dispersive element and recorded by a camera sensor. This allows the camera sensor to record a plurality of spectra, each with an associated spatial coordinate, along the longitudinal direction of the slit-shaped aperture. The device described is further configured to record further spectra along the longitudinal direction of the slit-shaped aperture in a direction different from the longitudinal direction of the slit-shaped aperture. The method underlying this disclosure for generating multispectral or hyperspectral images is also known as the so-called pushbroom method.

In addition to the pushbroom method, there are other methods for generating multispectral or hyperspectral images. In the so-called whiskbroom method, the region under study or else the object is scanned point by point and a spectrum is obtained for each point. In contrast, the staring method involves taking multiple images with the same spatial coordinates. Different spectral filters and/or illumination sources are used from image to image to resolve spectral information. Furthermore, there are methods according to which a two-dimensional multi-color image is broken down into a plurality of individual spectral images using suitable optical elements, such as optical slicers, lenses and prisms, which images are simultaneously acquired on different detectors or detector regions. This is sometimes referred to as the snapshot approach.

As described in DE 10 2020 105 458 A1, multispectral and hyperspectral imaging devices are particularly suitable as endoscopic imaging devices. In this context, multispectral and/or hyperspectral imaging is a fundamental field of application, for example for diagnostics and for assessing the success or quality of an intervention.

In addition, white light imaging is used in particular in the medical imaging field. Observed tissue is illuminated with white light and images of the tissue are generated using a camera or other image acquisition sensor system, which can then be displayed to a user.

Fluorescence imaging is also used, in particular in the medical imaging field. Tissue is specifically illuminated in a certain wavelength range in order to excite fluorescent dye molecules that have been specifically introduced into certain entities such as tissue regions. The subsequently emitted light with a longer wavelength can be observed through a suitably selected filter, by means of which the excitation light can be blocked.

Multimodal imaging devices allow the acquisition of, optionally, white light images and/or multispectral images and/or fluorescence images and/or hyperspectral images. Examples of such imaging devices are multimodal endoscopes and multimodal exoscopes. To realize different modes, light sources may be required that are operable in different illumination modes in order to generate illumination light in different spectral ranges, as required. Furthermore, switchable or interchangeable optical components may be required to adapt the imaging device to the different modes.

Based on the prior art, the object of the invention is to allow operation in different modes and, in particular, to achieve a high degree of efficiency and/or operating safety and simplicity.

This object is achieved according to the invention by an illumination device, an imaging device, a method for generating illumination light, and a method for operating an imaging device as described herein and defined in the claims.

An illumination device may be provided, in particular for providing illumination light for an imaging unit such as an endoscope, exoscope and/or microscope. It can be either an endoscope illumination device, an exoscope illumination device or a microscope illumination device.

In some embodiments, the illumination device comprises an optical interface for optically connecting an imaging unit, and an illumination unit that is designed to provide illumination light to the optical interface. The illumination unit can be multimodal and can comprise a plurality of luminous elements which are optionally activatable independently of one another and which are configured to emit light according to different emission spectra in order to provide the illumination light. The illumination unit may be operable in at least one multispectral mode in which a first group of the luminous elements is at least temporarily activated and in which the illumination unit provides illumination light for multispectral imaging. The illumination unit may further be operable in at least one fluorescence mode in which a second group of the luminous elements is at least temporarily activated and in which the illumination unit provides illumination light for fluorescence imaging. The luminous elements can comprise at least one luminous element which is included in both the first group and the second group.

Moreover, a method for generating illumination light for an imaging unit by means of an illumination device can be provided, in particular by means of an illumination device according to the invention. The illumination device comprises an optical interface for optically connecting an imaging unit, and an illumination unit that is designed to provide illumination light to the optical interface, wherein the illumination unit comprises a plurality of luminous elements which are optionally activatable independently of one another and which are configured to emit light according to different emission spectra in order to provide the illumination light. The method comprises the step of at least temporarily activating a first group of the luminous elements to provide illumination light for multispectral imaging and the step of at least temporarily activating a second group of the luminous elements to provide illumination light for fluorescence imaging. At least one of the luminous elements is activated at least temporarily both when the first group of luminous elements is activated at least temporarily and when the second group of luminous elements is activated at least temporarily.

In some embodiments, the illumination device can comprise an optical interface for optically connecting an imaging unit, and an illumination unit that is designed to provide illumination light to the optical interface. The illumination unit can comprise a plurality of luminous elements which are optionally activatable independently of one another and which are configured to emit light according to different emission spectra in order to provide the illumination light. The illumination unit may be operable in at least one multispectral mode in which a first group of the luminous elements that comprises at least two of the luminous elements is at least temporarily activated and in which the illumination unit provides illumination light for multispectral imaging. The luminous elements of the first group can be arranged such that light emitted by the luminous elements travels through a light path of at least substantially the same length from the relevant luminous element to the optical interface.

Furthermore, an imaging device can be provided, in particular a medical imaging device, which comprises an illumination device according to the invention and an imaging unit, for example an endoscope and/or exoscope and/or microscope, which can be connected to the optical interface of the illumination device.

Furthermore, a method for operating an imaging device is described herein, in particular an imaging device according to the invention which comprises an imaging unit. Here, illumination light generated according to a method according to the invention for generating illumination light is provided to the imaging unit.

In some embodiments, the imaging device can include an illumination device for providing illumination light for an imaging unit. The illumination device can comprise an optical interface for optically connecting an imaging unit, and an illumination unit that is designed to provide illumination light to the optical interface, wherein the illumination unit is multimodal and operable in a plurality of different illumination modes. An imaging device, in particular a medical imaging device, can comprise the illumination device and an imaging unit that can be connected to the optical interface of the illumination device, as well as a controller that is configured to automatically match an operating state of the imaging unit and an illumination mode of the illumination unit to one another. The controller can be configured to control the illumination device and/or the imaging unit.

Furthermore, a method for operating a medical imaging device can be provided which comprises automated matching of an operating state of the imaging unit and an illumination mode of the illumination unit.

The above features allow operation in different modes. In this process, a high degree of efficiency and/or operating safety and simplicity can be achieved. The proposed combination of imaging modes or luminous elements used for this purpose can reduce the complexity of light source and/or image acquisition. A small number of built-in luminous elements, filters and/or associated optical elements can be used while maintaining a wide range of functions. Furthermore, installation space can be saved, which allows a high degree of compactness to be achieved. By choosing light paths of substantially equal length, deviations and possible measurement errors can be avoided which are due to relative spectral intensities in different spectral ranges and which can occur, for example, when an endoscope (shaft) is rotated relative to the camera unit and/or when a light guide is rotated relative to the imaging unit. Due to the substantially equal length of the light paths, largely identical intensity profiles of the affected luminous elements can be achieved. Furthermore, by automatically matching the operating state of the imaging unit and the illumination mode, a high degree of operating comfort can be achieved and operating errors can be avoided. This makes it possible to provide a multimodal system that can be easily switched between different modes.

The imaging device can be a microscopic, macroscopic and/or exoscopic imaging device. The imaging unit can be configured as, and/or comprise, a microscope, macroscope and/or exoscope. In some embodiments, the imaging device can be an endoscopic imaging device, in particular an endoscope device. The imaging unit can be and/or comprise an endoscope. It shall be understood that the imaging unit may contain electronic components but may also be purely mechanical.

In some embodiments, the imaging device and, in particular, the imaging unit is configured to be insertable into a cavity for inspection and/or observation, for example into an artificial and/or natural cavity, such as into the interior of a body, into a body organ, into tissue or the like. The imaging device and, in particular, the imaging unit can also be configured to be insertable into a housing, casing, shaft, tube or other, in particular artificial, structure for inspection and/or observation.

The imaging device and, in particular, the imaging unit can be configured to acquire tissue parameters, images of wounds, images of body parts, etc. For example, the imaging device may be configured to image a surgical field. The imaging device and/or the imaging unit can comprise a spatially and spectrally resolving image acquisition unit which comprises at least one optical system and at least one image acquisition sensor system coupled to the optical system, which are configured to carry out image acquisition in which spatially and spectrally resolved image data are generated that comprise both spatial and spectral information.

The image acquisition unit and in particular the optical system and/or the image acquisition sensor system can be configured for multispectral and/or hyperspectral imaging, in particular for acquiring and/or generating multispectral and/or hyperspectral image data. Multispectral imaging or multispectral image data can refer in particular to such imaging in which at least two, in particular at least three, and in some cases at least five spectral bands can be acquired and/or are acquired independently of one another. Hyperspectral imaging or hyperspectral image data can refer in particular to imaging in which at least 20, at least 50 or even at least 100 spectral bands can be acquired and/or are acquired independently of one another.

The imaging device may operate according to the pushbroom method, and/or the whiskbroom method, and/or according to the staring method, and/or according to a snapshot principle.

In some embodiments, the imaging device and/or the imaging unit comprises a white light camera and/or sensor system for white light imaging. The imaging device and/or the imaging unit can be configured for white light imaging in addition to spectrally resolved imaging. A separate optical system and/or a common optical system can be used for this purpose. White light imaging and spectrally resolved imaging can be performed simultaneously or alternately, or sometimes simultaneously and sometimes sequentially.

In some embodiments, the imaging device and/or the imaging unit comprises a sensor system for fluorescence imaging. The imaging device and/or the imaging unit can be configured for fluorescence imaging in addition to spectrally resolved imaging and, if appropriate, in addition to white light imaging. A separate optical system and/or a common optical system can be used for this purpose. Fluorescence imaging, white light imaging (if appropriate) and spectrally resolved imaging can be performed simultaneously or alternately, or sometimes simultaneously and sometimes sequentially.

For some applications it can be advantageous to be able to use a high spectral resolution. Hyperspectral imaging is then recommended. It can be combined with white light imaging and/or fluorescence imaging. This makes real-time observation possible via a white light image and/or a fluorescence image, even if the acquisition of spectrally resolved image data only occurs substantially in real time, i.e., for example, several seconds are needed to create a spectrally resolved image.

For some applications it can be advantageous to generate spectral image data in real time. This includes, for example, the generation of a spectrally resolved image in less than a second or even several times per second. It can be useful to use multispectral imaging in this case. An optionally lower spectral resolution is then offset by a higher refresh rate. Depending on the application, it can be sufficient to consider only a few different spectral ranges and/or wavelengths, for example two or three or four or generally less than ten. In this case, additional white light imaging can optionally be omitted. Spectrally resolved image data that are acquired in real time or deliver several images per second can also be used for monitoring purposes, wherein it is not absolutely necessary to create a reproducible image for a user, but rather the image data can also be processed in the background.

The optical interface can be either detachable or connectable. In addition, the optical interface can be combined with a mechanical interface so that an optical connection is automatically established, for example, when the imaging unit is mechanically coupled.

The luminous elements can comprise single-color LEDs (light-emitting diodes) and/or laser diodes. Furthermore, at least one of the luminous elements can be a white light LED or other white light source. In some embodiments, the illumination unit comprises at least one blue luminous element, at least one red luminous element, at least one dark red luminous element and at least one near-IR luminous element (near-infrared luminous element), in particular LEDs or laser diodes in each case. In addition, the illumination unit can comprise at least one white light LED or other white light source.

The first group can comprise at least two luminous elements that emit spectrally differently. A high degree of efficiency in multispectral imaging can be achieved if the multispectral mode comprises different states in which a specific luminous element or a specific type of luminous element is activated at least temporarily. This allows targeted illumination in a specific spectral range, allowing different spectral images to be captured. Different luminous elements activated in different states can serve as different support points for multispectral imaging. At least one of these support points can be selected such that it is adapted to characteristic points of absorption spectra of physiologically relevant components, for example to an isosbestic point of the hemoglobin oxygenation curve. Multispectral imaging can additionally include the use of appropriate observation filters.

Furthermore, the second group can comprise at least two luminous elements that emit spectrally differently. The fluorescence mode can comprise different submodes and/or states in which a specific luminous element or a specific luminous element type is activated at least temporarily. This allows targeted excitation in a certain spectral range, so that fluorescence imaging can be carried out, for example, for a specifically selected dye. In other words, the at least one luminous element contained in both the first group and the second group can be used for both the multispectral mode and the fluorescence mode.

In some embodiments, the first group comprises only some but not all of the luminous elements. Alternatively or additionally, in some embodiments, the second group comprises only some but not all of the luminous elements. In the multispectral mode only luminous elements of the first group, in particular, are activated at least temporarily, whereas luminous elements that do not belong to the first group are deactivated. In the fluorescence mode only luminous elements of the second group, in particular, are activated at least temporarily, whereas luminous elements that do not belong to the second group are deactivated. In general, it shall be understood that the luminous elements can comprise different luminous element types and that exactly one luminous element of each of the different luminous element types, in particular, can be present. It shall be understood that mixed operating modes can also occur according to the invention, in which said modes are used sequentially. For example, multispectral imaging and fluorescence imaging can be performed sequentially.

The path of light that the light emitted by the luminous elements of the first group passes from the relevant luminous element to the optical interface extends in particular from a light-emitting surface of the relevant luminous element to a point on the optical interface at which light can be coupled out of the optical interface. A length of said light paths differs in particular by at most 20%, preferably by at most 10%, preferably by at most 5% and particularly preferably by at most 3%. These percentages may refer to the longest of the light paths being compared.

The operating state of the imaging unit, which is matched to the illumination mode, can define an imaging mode. For example, it may be an operating state in which multispectral imaging, white light imaging or fluorescence imaging can be performed. A versatile imaging device can be provided in particular if the controller for a multispectral operating state and/or for a fluorescence operating state and/or for a white light operating state sets the illumination device accordingly to a multispectral mode, fluorescence mode or white light mode.

The controller may effect automatic matching by electronically controlling the illumination device and/or the imaging unit. Matching can also be done inherently by choosing a suitable filter, for example an observation filter. The controller can be designed as a separate control unit or can be integrated in a controller of the imaging device.

Synergy with regard to the use of a luminous element for different modes and associated efficiency gains can be achieved in particular if at least one luminous element contained in both the first group and the second group emits light in the red spectral range, in particular in a spectral range between 600 nm and 680 nm, for example between 610 nm and 650 nm or between 620 and 660 nm or between 630 and 670 nm. The spectral range can be narrowband and cover the 660 nm wavelength. “Narrowband” may include a spectral width of at most 80 nm, in particular at most 40 nm or even at most 20 nm. This at least one luminous element can be configured to excite dyes absorbing in the red spectral range and to contribute to the illumination in the red spectral range for multispectral imaging.

In some embodiments, the illumination unit may be operable in at least one white light mode in which the illumination unit provides illumination light for white light imaging. The illumination light for white light imaging can be broadband white light. Alternatively, the illumination light for white light imaging can comprise a plurality of narrow wavelength bands that are separated from one another, for example a blue, a red and a dark red band. “Dark red” is to be understood as having a “longer wavelength than red” and refers to the spectral position, but not to light intensity. The illumination light for white light imaging can be a mixture of light from different luminous elements.

In the white light mode, a third group of luminous elements may be activated at least temporarily to provide the illumination light for white light imaging. The luminous elements can comprise at least one luminous element which is contained both in the first group and/or second group and in the third group. In some cases, the third group may comprise only some but not all of the luminous elements. In the white light mode only luminous elements of the third group, in particular, are activated at least temporarily, whereas luminous elements that do not belong to the third group are deactivated. In other words, the illumination unit may comprise luminous elements that serve one, two or all three of the aforementioned illumination modes. This allows for a plurality of luminous elements to be used multiple times.

At least one luminous element contained both in the first group and/or second group and in the third group can emit light in the red spectral range, in particular in a spectral range between 600 nm and 680 nm, for example between 610 nm and 650 nm or between 620 and 660 nm or between 630 and 670 nm. The advantages of using luminous elements together are particularly evident when at least one red luminous element can be used for all three modes.

At least one luminous element contained both in the first group and/or second group and in the third group can emit light in the blue spectral range, in particular in a spectral range between 440 and 480 nm. At least one blue luminous element can be conveniently used both in the fluorescence mode and in the white light mode.

Generally speaking, the luminous elements, as mentioned, can comprise at least one, in particular blue, luminous element which emits light in a spectral range between 440 and 480 nm. Moreover, the luminous elements, as mentioned, can comprise at least one, in particular red, luminous element which emits light in a spectral range between 600 and 680 nm, for example between 610 and 650 nm or between 620 and 660 nm or between 630 and 670 nm. Alternatively or additionally, the luminous elements can comprise at least one, in particular dark red, luminous element which emits light in a spectral range between 750 and 790 nm. Alternatively or additionally, luminous elements can comprise at least one, in particular near-IR emitting, luminous element that emits light in a spectral range between 920 and 960 nm. In addition, the luminous elements can include a white light luminous element. A compact and versatile illumination unit can be provided in particular if at least one luminous element of each of the above-mentioned luminous element types is present. For example, in fluorescence mode, the blue and the red luminous element can be used, and in the case of suitable dyes, the dark red luminous element may also be used. In multispectral mode, the dark red and near-IR emitting luminous elements can be used. In white light mode, the white light luminous element can be used. In white light mode, it can be supplemented by the blue luminous element and, if necessary, the red luminous element. This makes it possible to supplement wavelength ranges by means of colored luminous elements in which the white luminous element provides a reduced intensity, for example due to its construction but in particular due to filters and optical elements of the illumination unit. In addition, the colored luminous elements can be used to adjust a color temperature for white light imaging.

In some embodiments, the second group comprises a single luminous element and/or a single type of luminous elements. For example, a white light luminous element, a red luminous element and an IR-emitting luminous element can be provided, with particular reference being made to the above values as regards possible spectral ranges. The first group can then, for example, comprise the red and the IR-emitting luminous element. The second group can comprise the IR-emitting luminous element, in particular as the only luminous element or as the only type of luminous element.

A favorable arrangement of luminous elements is made possible in particular if the illumination unit comprises at least one crossed beam splitter, by means of which light can be deflected from opposite input sides to an output side, wherein at least one of the luminous elements is arranged on the opposite input sides of the crossed beam splitter. In some embodiments, two or more crossed beam splitters may be provided that are arranged optically one behind the other. The at least one crossed beam splitter can comprise two beam splitter elements, the transmittance of which is adapted to the respectively associated luminous element. The beam splitter elements each comprise, in particular, a notch filter so that they reflect in a narrow spectral band but otherwise transmit. The spectral position and/or width of the corresponding notch can be adapted to the spectral range of the respectively associated luminous element, so that its light is redirected, but light from other luminous elements is at least largely transmitted.

In some embodiments, the luminous elements can comprise at least four narrowband emitting single-color luminous elements, each with different spectral ranges, and at least one broadband emitting white light luminous element. In this regard, reference is also made to what has been said above in connection with colored luminous elements. In combination with two crossed beam splitters, one of the single-color luminous elements can be assigned to one of the beam splitter elements of the two beam splitters. Furthermore, the white light luminous element can be arranged on a distal side of the two beam splitters as viewed from the optical interface, so that light from the white light luminous element is coupled through both beam splitters in the direction of the optical interface.

A wide range of functions in combination with a compact design and the utilization of synergy effects when using luminous elements can be achieved in particular if the illumination unit is operable in at least one hyperspectral mode in which a plurality of luminous elements is activated, the emission spectra of which together cover at least a spectral range from 450 nm to 850 nm, and in which the illumination unit provides illumination light for hyperspectral imaging. This may in particular include all of the luminous elements.

It shall be understood that, in particular when using laser diodes, suitable polarization filters can be used for the optical filters mentioned herein. Furthermore, in particular when using laser diodes, at least one crossed beam splitter can be used, the beam splitter elements of which are provided with polarization filters. Selective transmittance can then be achieved by combining different polarizations.

In some embodiments, the illumination unit can define a common optical path into which emitted light from the luminous elements can be coupled. The luminous elements of the first group can each have a light-emitting surface, wherein the light-emitting surfaces of the luminous elements of the first group are arranged equidistantly with respect to the common optical path. The optical path can be defined by the at least one crossed beam splitter. In particular, the optical path can extend from an output coupling point of the beam splitter closest to the optical interface to the optical interface.

A space-efficient arrangement with high luminous efficacy can be achieved in particular if the crossed beam splitter is arranged such as to couple light coming from the opposite input sides into the common optical path. The light paths of substantially equal length can be achieved by ensuring that a distance from the crossed beam splitter to the opposite luminous elements assigned to it is substantially equal.

The at least one beam splitter can comprise at least three input sides, two of which form opposite input sides and a third of which is opposite an output side. In some embodiments, the illumination unit can comprise at least two crossed beam splitters arranged optically one behind the other. If a plurality of beam splitters is present, they can be arranged such that an output side of a first beam splitter faces an input side of a second beam splitter. A luminous element, in particular the white light luminous element, can be arranged on the input side of a beam splitter which is furthest away from the optical interface.

The imaging unit can comprise a filter unit with optical filters that can be switched at least between a multispectral mode and a fluorescence mode. This allows the imaging unit to be adaptable to the multifunctionality of the illumination unit. The fluorescence mode of the filter unit can comprise a plurality of submodes defined by different filters. For example, different edge filters can be used which absorb/block the respectively used spectrum of the corresponding luminous element used for excitation and at least substantially only transmit fluorescent light.

The imaging unit can have a stereoscopic ocular, wherein the ocular comprises two ocular sides in which different filters are installed. For example, one ocular side can comprise a filter for multispectral imaging and one ocular side can comprise a filter for fluorescence imaging. This makes it easy to generate or view parallel representations of multispectral images and fluorescence images.

The illumination mode of the illumination unit and/or the operating state of the imaging unit can be specified by at least one user action. The user action may include, for example, selecting an illumination mode, selecting an imaging mode, selecting a particular optical filter, changing an exchangeable shaft, or the like. The controller can be configured to automatically match, in response to the user action, the operating state of the imaging unit and the illumination mode of the illumination unit to one another. This allows for intuitive operability and prevents at the same time operating errors because necessary adjustments can be made automatically. The controller can be configured to adjust the operating state of the imaging unit when the user action changes the illumination mode. Alternatively or additionally, the controller can be configured to adjust the illumination mode when the user action changes the operating state of the imaging unit.

The imaging unit can comprise a camera unit, wherein the controller is configured to adjust an illumination mode of the illumination unit on the basis of an operating state of the camera unit. The camera unit can comprise an imaging sensor system and/or optical filters. The operating state of the camera unit depends in particular on a selection of an optical filter, which selection can be made by a user.

An efficient and safe operating concept can be provided in particular if the camera unit comprises a plurality of optical filters that can optionally be introduced into an observation beam path of the camera unit and that define different observation modes that can be selected by a user. The controller can be configured to adjust the illumination mode on the basis of a selected observation mode. For example, the camera unit can comprise different filters for fluorescence imaging and for multispectral imaging. If the user introduces a specific filter into the observation beam path, the system immediately switches to the corresponding mode. With a single adjustment, all components can be correctly adjusted to match one another.

In some embodiments, the optical filters can be manually introduced into the observation beam path by the user. The camera unit can then comprise at least one filter sensor which is configured to automatically detect an optical filter currently introduced into the observation beam path and to generate a sensor signal which contains information relating to the detected optical filter. The controller can be configured to detect the observation mode of the camera unit in accordance with the sensor signal. This allows a user to set the imaging device to the desired state by simple manual handling without having to make any special adjustments to the illumination unit. The filter sensor can be configured to directly detect the introduced optical filter, for example optically. The introduced optical filter can be detected particularly easily if the available optical filters are mounted on a movable filter carrier, such as a filter wheel or a slider, and the sensor is a position sensor that detects a position of the filter carrier. In that case, equipment of the filter carrier can be stored and/or storable in the controller. The controller can be configured to determine the corresponding optical filter based on the detected position.

Alternatively or additionally, the camera unit can comprise an automated filter unit which is configured to automatically introduce at least one of the optical filters into the observation beam path in accordance with an observation mode specified by a user. The imaging device can have a user interface via which the user can select the optical filter. The user interface can, for example, have push buttons, touch-sensitive elements, a display, a touch display or other input means. In some embodiments, a filter selection entered by the user can be immediately interpreted by the controller to adjust the modes of the illumination unit and imaging unit such as to match the filter selection made.

The imaging unit can have a distal shaft, wherein the camera unit is a proximal camera unit, and wherein the shaft is optically coupled to the camera unit. The camera unit can be designed separately from the shaft. In particular, in that case the components of the camera unit are arranged outside the shaft. This is useful, for example, if the imaging unit is an endoscope. The shaft can comprise optical elements that guide light from a distal end of the shaft to a proximal end of the shaft. The proximal end of the shaft can be optically coupled to the camera unit. The shaft thus guides light to and from an imaged region, whereas actual image acquisition takes place proximal to the shaft in the camera unit.

A high degree of versatility can be achieved in particular if the imaging unit comprises a broadband transmitting optics that can be used uniformly in the different illumination modes. This allows a single imaging unit, in particular a single endoscope, to be used for different spectral ranges. It is then not necessary to use a separate optics for each application. For example, the broadband transmitting optics can be an imaging optics. It can be transmissive at least in a range between 400 nm and 1000 nm.

In some embodiments, the imaging unit comprises a proximal base unit to which different exchangeable shafts designed for different observation modes can optionally be optically and electronically coupled. The controller can be configured to adjust an illumination mode of the illumination unit on the basis of the observation mode defined by a currently coupled exchangeable shaft. This allows systems with exchangeable shafts to intuitively switch between different modes without the need for user adjustments. The necessary settings of the components are made in response to the selection of a specific exchangeable shaft. The user only has to connect the exchangeable shaft and thus has already selected a specific imaging mode and associated illumination mode. The different exchangeable shafts can comprise an image acquisition sensor system, such as a Tipcam. This may be a camera and/or camera arrangement and/or camera sensor arranged in a distal end region and/or at a distal end of the relevant exchangeable shaft. A proximal camera unit can be dispensed with in these embodiments. In particular, the camera unit can be partially or completely integrated in the exchangeable shaft or defined by selecting a specific exchangeable shaft. The base unit can be free of an image acquisition sensor system.

The imaging device can be part of a medical imaging system. It can comprise at least two different exchangeable shafts, which can be optionally connected to the base unit of the imaging unit. The exchangeable shafts can each comprise an integrated camera and/or integrated optical filters. The integrated cameras and/or integrated filters may vary from exchangeable shaft to exchangeable shaft. For example, an exchangeable shaft for white light imaging and/or an exchangeable shaft for fluorescence imaging and/or an exchangeable shaft for multispectral imaging may be provided.

The devices and systems according to the invention and the methods according to the invention should not be limited to the application and embodiment described above. In particular, they can have a number of individual elements, components and units as well as method steps, which differ from a number mentioned herein, in order to fulfill a function described herein. In addition, for the ranges of values specified in this disclosure, values within the stated limits shall also be deemed to be disclosed and to be usable in any manner.

It is in particular pointed out that all features and properties described with regard to a device, but also procedures, can be analogously transferred to methods and can be used within the meaning of the invention and are considered to be co-disclosed. The same applies in the opposite direction. This means that structural features mentioned in relation to methods, i.e., features relating to the device, can also be taken into account, claimed and also counted as part of the disclosure within the scope of the device claims.

The present invention is described below by way of example with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using numerical words, such as first, second, third object, etc., these are used to name and/or assign objects. Accordingly, for example, a first object and a third object may be included, but not a second object. However, a number and/or sequence of objects could also be derived using numerical words.

In the drawings:

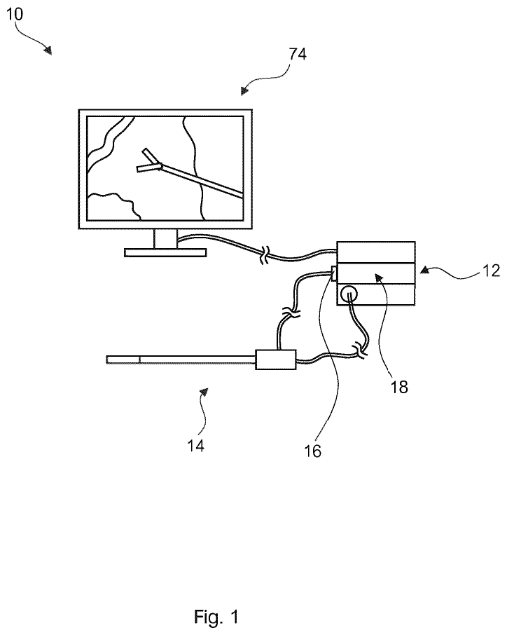

FIG. 1 shows a schematic representation of an imaging device having an illumination device;

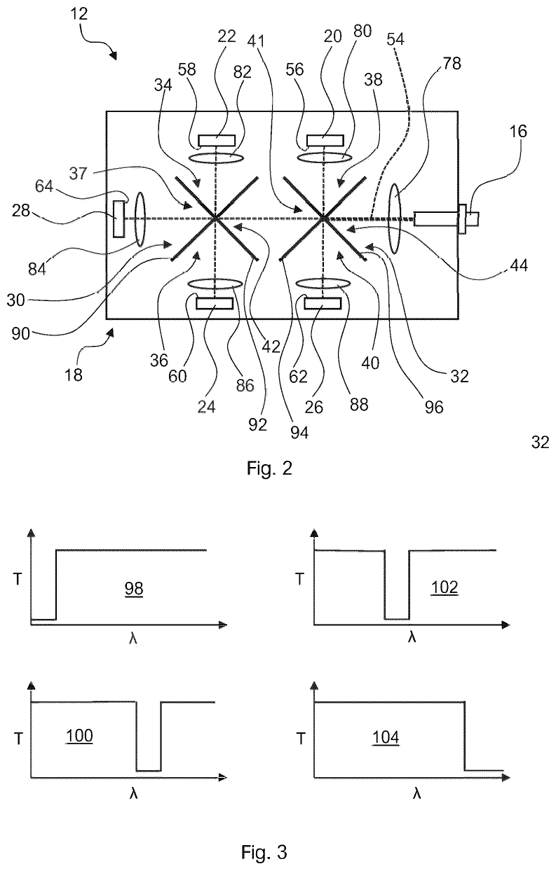

FIG. 2 shows a schematic representation of the illumination device;

FIG. 3 shows schematic transmission curves of beam splitter elements of the illumination device;

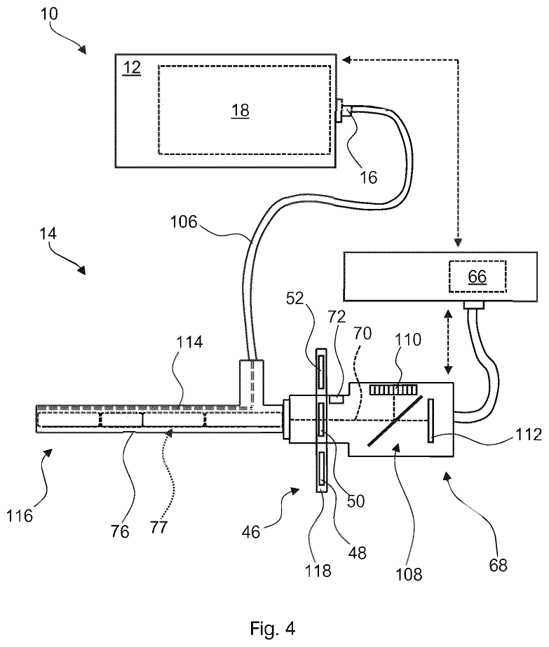

FIG. 4 shows a schematic representation of the imaging device;

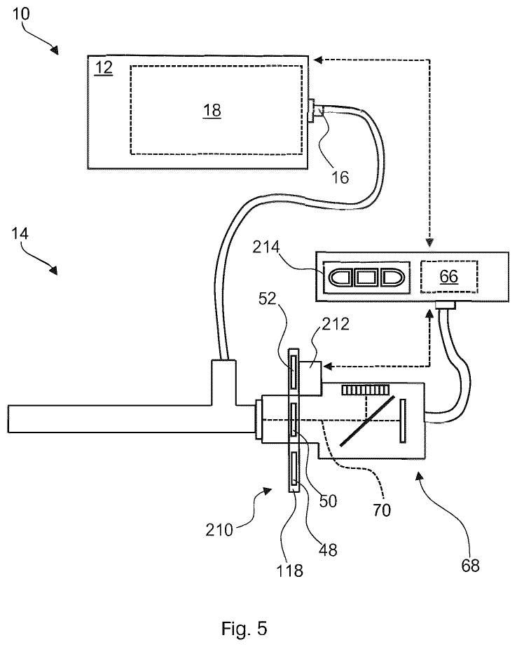

FIG. 5 shows a schematic representation of another embodiment of the imaging device;

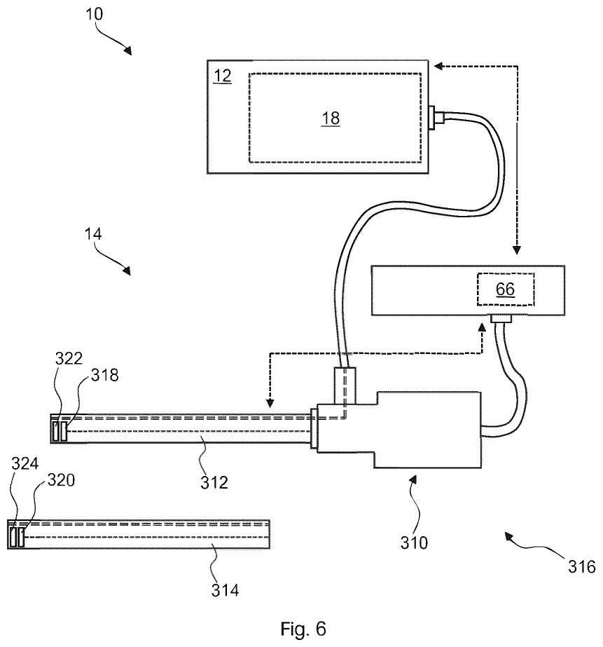

FIG. 6 shows a schematic representation of yet another embodiment of the imaging device;

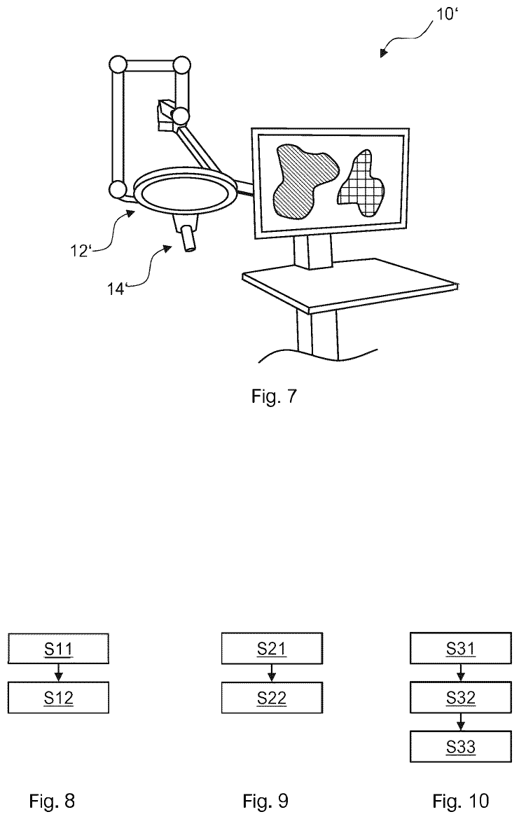

FIG. 7 shows a schematic perspective representation of another embodiment of the imaging device;

FIG. 8 shows a schematic flow chart of a method for generating illumination light for an imaging unit by means of an illumination device;

FIG. 9 shows a schematic flow chart of a method for operating an imaging device; and

FIG. 10 shows a schematic flow chart of a method for operating an imaging device.

FIG. 1 shows a schematic representation of an imaging device 10. In the instance shown by way of example, the imaging device 10 is an endoscopic imaging device, specifically an endoscope device. Alternatively, the imaging device 10 could be an exoscopic, a microscopic or a macroscopic imaging device. The imaging device 10 is shown by way of example as a medical imaging device. The imaging device 10 is provided, for example, for examining a cavity.

The imaging device 10 comprises a medical imaging unit 14. In the shown case, this is an endoscope.

The imaging device 10 further comprises an illumination device 12 having an optical interface 16, and an illumination unit 18. The imaging unit 14 can be optically connected to the optical interface 16. The optical interface 16 can be part of an optical-mechanical interface that can be optionally detachable and connectable. The illumination apparatus 14 can optionally be decoupled from the illumination device 12. The illumination unit 18 is designed to provide illumination light to the optical interface 16. When imaging by means of the imaging unit 14, the illumination unit 18 can accordingly provide the required illumination light, which is guided to the illumination apparatus 14 and from there coupled out onto an object to be imaged, such as a situs.

In the illustrated case, the imaging device 10 further comprises a display unit 74 on which images can be displayed that are based on image data acquired by means of the imaging unit 14. These can be video images, still images, overlays of different images, partial images, image sequences, etc.

The imaging device 10 is multimodal. By way of example, the imaging device can be operated in three basic modes: a multispectral mode, a fluorescence mode and a white light mode. It can further be provided for the imaging device 10 to be operable in a hyperspectral mode in addition or as an alternative to the multispectral mode.

The illumination device 12 is multimodal. The illumination device 12 is operable in different illumination modes in which it provides light for different imaging modes. Here, the illumination device 12 is operable in three basic modes, namely a multispectral mode, a fluorescence mode and a white light mode. The imaging unit 14 is also operable in different operating modes, specifically also in at least a multispectral mode, a fluorescence mode and a white light mode. In the corresponding operating mode of the imaging device 10, the modes of the illumination device 12 are matched to one another.

FIG. 2 shows a schematic representation of the illumination device 12. The illumination unit 18 comprises a plurality of independently activatable luminous elements 20, 22, 24, 26, 28. They are configured to emit light according to different emission spectra in order to provide illumination light, i.e., the relevant emission spectrum differs from luminous element to luminous element.

The luminous elements 20, 22, 24, 26, 28 are designed as LEDs, for example. Specifically, a first luminous element 20 is configured as a red LED, a second luminous element 22 as a dark red LED, a third luminous element 24 as a blue LED and a fourth luminous element 26 as a near-IR LED. The colored luminous elements 20, 22, 24, 26 each emit in a narrow band, for example with an emission peak for example at wavelengths 660 nm (first luminous element 20), 770 nm (second luminous element 22), 460 nm (third luminous element 24) and 940 nm (fourth luminous element 26).

A fifth luminous element 28 is further provided, which in the present case is a white light luminous element, for example a white light LED. The fifth luminous element 28 emits, for example, in a spectral range of about 400 to 700 nm. In other embodiments, laser diodes can also be used, in particular as colored luminous elements.

Depending on the illumination mode, some of the luminous elements 20, 22, 24, 26, 28 are activated at least temporarily, whereas other luminous elements 20, 22, 24, 26, 28 may not be used in the illumination mode in question.

In the present case, a first group comprises the first luminous element 20 and the fourth luminous element 26. The first group may additionally comprise luminous element 22 and/or luminous element 24. The first group is used for multispectral imaging, with the contained luminous elements 20, 26 and, if applicable, 22 and 24 each serving as support points. In multispectral mode, for example, the first luminous element 20 is initially used for illumination and an image is taken. Then, the fourth luminous element 26 is used for illumination and an image is taken. The images are based on remission, i.e., the light scattered back from the object being imaged is considered. The two different support points can be used to obtain spectral information about the object to be imaged. For example, certain tissue types, a perfusion state, a tissue condition or the like can be assessed in this way.

Furthermore, a second group comprises the first luminous element 20, the second luminous element 22 and the third luminous element 24. The second group is used for illumination in fluorescence imaging. For example, objects that have been colored with appropriately selected dyes can be viewed. Also, different dyes can be introduced into different types of tissue or the like, which are viewed simultaneously. By specifically exciting a particular dye, it is excited to fluoresce. The fluorescent light is then imaged. The first luminous element 20 is suitable, for example, for exciting the dye cyanine 5.5 (Cy 5.5). The second luminous element 22 is suitable for exciting the dye indocyanine green (ICG). The third luminous element 24 is suitable for exciting the dye fluorescein.

Furthermore, a third group comprises the fifth luminous element 28. In the present embodiment, the third group moreover comprises the first luminous element 20 and the third luminous element 24. The third group is used to provide illumination light for white light imaging. For this purpose, white light from the fifth luminous element 28 can be mixed with light from certain colored luminous elements, whereby spectral losses can be compensated and/or a color temperature can be specifically adjusted.

It can be seen that some of the luminous elements 20, 22, 24, 26, 28 are assigned to a plurality of groups, for example the first luminous element 20 to all three groups and the third luminous element 24 and possibly also the second luminous element 22 to the second group and third group.

Alternatively or additionally, it may also be provided for some or all of the luminous elements 20, 22, 24, 26, 28 to be used in a hyperspectral mode. A broad spectrum of excitation is then generated. In combination with a suitable hyperspectral detector, spectral information about the object to be imaged can then be captured across the entire visible and near-IR spectrum. For this purpose, the imaging unit 14 may comprise a pushbroom arrangement as a hyperspectral detector. In other embodiments, a whiskbroom arrangement, a staring arrangement and/or a snapshot arrangement is used. The imaging unit 14 can be a hyperspectral imaging unit. Regarding different methods of hyperspectral imaging and components required for this, reference is made to the article “Review of spectral imaging technology in biomedical engineering: achievements and challenges” by Quingli Li et al., published in Journal of Biomedical Optics 18(10), 100901, October 2013, and to the article

“Medical hyperspectral imaging: a review” by Guolan Lu and Baowei Fei, published in Journal of Biomedical Optics 19(1), 010901, January 2014.

The illumination unit 18 comprises two crossed beam splitters 30, 32. They each comprise an output side 42, 44, an input side 37, 41 opposite the output side 42, 44 and two input sides 34, 36, 38, 40 opposite one another. All input sides 34, 36, 37, 38, 40, 41 lead incident light to the corresponding output side 42, 44. The output side 42 of a first crossed beam splitter 30 faces an input side 41 of the second crossed beam splitter 32. The output side 44 of the second crossed beam splitter 32 faces the optical interface 16. The two crossed beam splitters 30, 32 are preferably arranged coaxially to one another and/or to the optical interface.

The illumination unit 18 can comprise suitable optical elements such as lenses and/or mirrors (not shown). A plurality of lenses 78, 80, 82, 84, 86, 88 is shown by way of example in FIG. 2. A lens 78 is assigned, for example, to the optical interface 16 and couples light coming from the output side 44 of the second crossed beam splitter 32 into the optical interface 16. Furthermore, each of the luminous elements 20, 22, 24, 26, 28 can be assigned a lens 80, 82, 84, 86, 88. A particularly high degree of compactness can be achieved in particular if the luminous elements 20, 22, 24, 26, 28 are each arranged without an intermediate mirror on input sides 34, 36, 37, 38, 40 of the at least one crossed beam splitter 30, 32. The luminous elements 20, 22, 24, 26, 28 can then be moved very close to the at least one crossed beam splitter 30, 32.

The crossed beam splitters 30, 32 each comprise two beam splitter elements 90, 92, 94, 96. They can basically be partially transmitting, so that light from all input sides 34, 36, 37, 38, 40, 41 is redirected to the relevant output side 42, 44. In the present embodiment, the beam splitter elements 90, 92, 94, 96 are selectively light-transmissive. This is illustrated with further reference to FIG. 3. The beam splitter elements 90, 92, 94, 96 can be filters that reflect only in a defined region, but otherwise have high transmission. FIG. 3 shows transmission curves 98, 100, 102, 104 of the beam splitter elements 90, 92, 94, 96 of the two crossed beam splitters 30, 32. Each of the colored luminous elements 20, 22, 24, 26 or each of the opposite input sides 34, 36, 38, 40 is assigned one of the beam splitter elements 90, 92, 94, 96. The beam splitter elements 90, 92, 94, 96 are selected such as to each reflect in the wavelength range in which the associated luminous element 20, 22, 24, 26 emits, but to otherwise largely transmit. For this purpose, notch filters can be used in the medium wavelength range, which can have, for example, transmission spectra 100 and 102. At spectral edges, high-pass or low-pass filters can be used instead of notch filters (see transmission spectra 98 and 104).

Due to the specific transmission spectra 98, 100, 102, 104 of the crossed beam splitters 30, 32, light from the fifth luminous element 28 is spectrally clipped. It may therefore be expedient, in the manner already mentioned, to supplement the light blocked by the beam splitters 30, 32 in a targeted manner by means of the luminous elements 20 and 24, if necessary also 22 and/or 26. This allows for supplementing specifically in those spectral ranges in which the beam splitters 30, 32 absorb and/or reflect light from the fifth luminous element 28, but in any case do not transmit it to the optical interface 16. The additionally used luminous elements 20, 24 and, if applicable, 22 are preferably operated with reduced power or with adjusted power. The aim here may be to at least largely restore the original spectrum of the fifth luminous element 28.

In some embodiments, the fifth luminous element 28 may alternatively be a green luminous element or, more generally, a colored luminous element that emits primarily in the spectral range that the at least one beam splitter 30, 32 transmits. For example, in such embodiments, the fifth luminous element 26 may be an LED having an emission peak at about 530 nm. A green laser diode can also be used for this purpose. Here, it can be provided that color mixing takes place in white light mode and, in particular, that no individual white light source such as a white light LED is used, but that white light from separate luminous elements is specifically mixed.

It shall be understood that in case of suitable dyes, such a green luminous element can also be used in fluorescence mode. Alternatively or additionally, it could be used in multispectral mode.

The illumination unit 18 defines a common optical path 54 into which emitted light from the luminous elements 20, 22, 24, 26, 28 can be coupled. The common optical path 54 extends from the output side 44 of the second crossed beam splitter 32 to the optical interface. In the present case, the common optical path 54 is arranged coaxially with the fifth luminous element 26.

In the embodiment shown, the luminous elements 20, 26 of the first group are arranged such that light emitted by the luminous elements 20, 26 travels through a light path of at least substantially the same length from the relevant luminous element 20, 26 to the optical interface 16. The luminous elements 20, 26 of the first group each have a light-emitting surface 56, 58. The light-emitting surfaces 56, 62 are arranged equidistantly with respect to the common optical path 54. In the present case, this is achieved in that the two luminous elements 20, 26 are arranged at the same distance from the beam splitter 32 assigned to them (in the present case, by way of example, the second beam splitter 32), in particular from its opposite input sides 38, 40. The light is coupled by the crossed beam splitter 32 into the common optical path 54.

The beam splitters 30, 32 are in particular arranged such that light-emitting surfaces 56, 58, 60, 62, 64 of the luminous elements 20, 22, 24, 26, 28 are each arranged equidistantly with respect to their associated crossed beam splitter 30, 32.

By using crossed beam splitters 30, 32 and luminous elements 20, 22, 24, 26, 28 that can be used together for different modes, the illumination unit 18 or the illumination device 12 has a high degree of compactness. In addition, the equidistant arrangement ensures that no spectral shifts occur when the imaging unit 14 or its light guide is rotated relative to the optical interface 16.

It shall be understood that a different number of luminous elements 20, 22, 24, 26, 28 and/or a different number of crossed beam splitters 30, 32 can be used. The use of crossed beam splitters 30, 32 has proven to be particularly useful. In other embodiments, however, other types of beam splitters and/or other optical elements can be used to couple light from the luminous elements 20, 22, 24, 26, 28 into the optical interface 16.

FIG. 4 shows a schematic representation of the imaging device 10. The imaging unit 14 is optically coupled to the optical interface 16, for example via a light guide 106 such as at least one optical fiber.

The imaging device 10 has a controller 66 which is configured to automatically match an operating state of the imaging unit 14 and an illumination mode of the illumination unit 18 to one another. In the present case, a user can specify the operating mode of the imaging unit 14 by means of a user action. The controller 66 then adjusts the appropriate illumination mode of the illumination unit 18. Alternatively or additionally, the user can adjust a specific illumination mode of the illumination unit 18 by means of a user action. The controller 66 can then adjust an appropriate operating mode of the imaging unit 14. The illumination device 12 and/or the imaging device 10 has, for example, a user interface via which the user can enter corresponding commands.

The imaging unit 14 comprises a camera unit 68 and a distal shaft 76. The distal shaft 76 is optically coupled to the camera unit 68. The camera unit 68 can have a connection for the distal shaft 76, wherein the distal shaft 76 can be optionally decouplable and couplable. The distal shaft 76 can also be permanently optically and/or mechanically coupled to the camera unit 68. The camera unit 68 is arranged proximally with respect to the shaft 76. The camera unit 68 comprises an imaging sensor system 108, in the present case, for example, a white light sensor 110 and a near-IR sensor 112. Generally speaking, the imaging sensor system 108 can have one or more at least spatially resolving light sensors/image sensors, for example at least one CMOS sensor and/or at least one CCD sensor. The shaft 76 comprises optical elements (not shown) by means of which light can be guided to the camera unit 68 in order to be able to optically capture the object to be imaged. The shaft 76 further comprises at least one light path 114, for example defined by a light guide such as an optical fiber, which leads to a distal portion 116 of the shaft 76 and by means of which the illumination light originating from the optical interface 16 of the illumination device 12 can be coupled out to the object to be imaged.

The camera unit 68 has different operating states, specifically for example at least one multispectral operating state and one fluorescence operating state and, in the present embodiments, additionally a white light operating state and possibly a hyperspectral operating state. The controller 66 automatically adapts the illumination mode of the illumination unit 18 to the current operating state of the camera unit 68. The controller 66 can set the image recording behavior of the camera unit 68. For example, the controller 66 can adjust the exposure time, sensitivity/amplification/gain and/or other operating parameters of the camera unit 68 or, in particular, its image acquisition sensor system 108 and, if applicable, its optics, thereby defining different operating states of the imaging unit 14. In the present case, the controller 66 triggers the illumination unit 18 synchronously with the camera.

The imaging unit 14 comprises a filter unit 46 with optical filters 48, 50 52. Three optical filters are shown as an example, but it shall be understood that a different number can be used. The filter unit 46 can be switched between a multispectral mode and a fluorescence mode. Moreover, the filter unit 46 can additionally be switchable to a white light mode and/or a hyperspectral mode. The optical filters 48, 50, 52 can optionally be introduced into an observation beam path 70 of the camera unit 68, thereby defining different observation modes. In the present case, they define the operating states of the camera unit 68.

A basic imaging mode can be associated with a plurality of optical filters 48, 50, 52. In particular for fluorescence imaging, a different suitable optical filter can be used depending on the luminous element 20, 22, 24, 26, 28 used for excitation. For example, in the present case the first luminous element 20 (red) is combined with an optical filter that transmits wavelengths greater than 730 nm but blocks shorter wavelengths. This makes it possible in particular to ensure that only fluorescent light and not the excitation light itself is detected. For example, said optical filter can absorb at least in the 600 nm to 730 nm range. Furthermore, in the present case, for example, the second luminous element 22 (dark red) is combined with a filter which absorbs in the 700 to 850 nm range or which only transmits significantly above 850 nm.

The user can select a specific filter 48, 50, 52 and thereby directly selects an associated observation mode or operating state of the camera unit 68. For this purpose, the camera unit 68 has a filter sensor 72 which can automatically detect an optical filter currently introduced into the observation beam path 70. The user can thus manually introduce a selected filter 48, 50, 52 into the observation beam path 70. In the example shown, the optical filters 48, 50, 52 are mounted on a filter carrier 118. Said filter carrier 118 can be moved into different positions, whereby one of the optical filters 48, 50, 52 can be selected in each case. The filter sensor 72 then detects the currently selected optical filter 48, 50, 52. The controller can then determine the current operating state of the camera unit 68 and thus of the imaging unit 14 based on a sensor signal from the filter sensor 72 and automatically adapt the illumination mode of the illumination unit 18 accordingly. The user thus puts the entire imaging device 10 into the desired mode by a simple user action such as a manual selection of an optical filter 48, 50, 52. A user can basically combine different filters with different illumination modes to create different types of contrast.

In the illustrated case, the imaging unit 14 and in particular the shaft 76 comprises a broadband transmitting optics 77, which can be used uniformly in the different illumination modes. In the present case, the broadband optics 77 is designed for a spectral range of at least 400 nm to 1000 nm. It can be used uniformly for different illumination and/or observation spectral ranges.

In some embodiments, the imaging unit 14 can be configured as a stereoendoscope that comprises a stereoscopic ocular having two sides. Different optical filters can be connected upstream of these sides independently of one another, allowing different contrast images to be superimposed on one another.

In the following, in the context of further embodiments and modifications, the same reference signs as above will be used for identical or similar components. As regards their description, reference is generally made to the above embodiments, whereas primarily the differences between the embodiments will be explained below. Also, reference signs have been partially omitted in the following figures for reasons of clarity.

FIG. 5 shows a schematic representation of another embodiment of the imaging device 10. The imaging device 10 comprises an illumination device 12 having an optical interface 16, and an illumination unit 18 as well as an imaging unit 14 which is connected to the optical interface 16. The imaging unit 14 comprises a camera unit 68 having an automated filter unit 210. The automated filter unit 210 comprises a plurality of optical filters 48, 50, 52, which can be automatically introduced into an observation beam path 70 of the camera unit 68 in accordance with an observation mode specified by a user.

The automated filter unit 210 comprises a filter drive 212 which is configured to automatically move the optical filters 48, 50, 52 into or out of the observation beam path 70. The optical filters 48, 50, 52 can be mounted on a filter carrier 118 which is connected to the filter drive 212. The filter drive 212 can be configured to move, for example to shift and/or rotate and/or pivot, the filter carrier 118.

The imaging unit 14 has a user interface 214 by means of which the user can adjust a desired observation mode. For example, a desired position of the filter carrier 118 can be specified by means of the user interface 214.

The imaging unit 14 further has a controller 66. The controller 66 is coupled to the filter drive 212 and the user interface 214. The controller 66 is configured, in particular, to process a user specification of an observation mode and to control both the filter unit 210 and the illumination unit 18 in accordance with said user specification. The controller 66 can thus adjust an operating state of the imaging unit 14 and an illumination mode of the illumination unit 18 matched thereto in accordance with an observation mode selected by the user.

FIG. 6 shows a schematic representation of yet another embodiment of the imaging device 10. The imaging device 10 comprises an illumination device 12 having an optical interface 16, and an illumination unit 18 as well as an imaging unit 14 which is connected to the optical interface 16. The imaging unit 14 comprises a proximal base unit 310. The proximal base unit 310 is connected to the optical interface 16 of the illumination device 12. Illumination light generated by the illumination device 12 can thus be supplied to the proximal base unit 310. The imaging unit 14 further comprises a controller 66, which in some embodiments may be integrated in the base unit 310.

Optionally, different exchangeable shafts 312, 314 can be optically and electronically coupled to the proximal base unit 310. The base unit 310 has an interface 316 for coupling different exchangeable shafts 312, 314. Said interface 316 supplies the illumination light coming from the illumination device 12 to a coupled exchangeable shaft 312, 314. The interface 316 is further configured to electrically supply a coupled exchangeable shaft 312, 314 and/or to electronically connect it to the controller 66 of the imaging unit 14.

The exchangeable shafts 312, 314 each have an integrated camera 318, 320 and integrated optical filters 322, 324. The integrated cameras 318, 320 are configured as Tipcams. In the present case, the integrated camera 318 of a first exchangeable shaft 312 is configured for multispectral imaging. Furthermore, the integrated camera 310 of a second exchangeable shaft 314 is configured for fluorescence imaging. The optional optical filters 322, 324 can be adapted thereto.

In other embodiments, it is also possible to use exchangeable shafts that only include optical filters but no integrated camera. In that case, they can be couplable to a proximal camera unit. In some cases, the proximal camera unit can then be designed without an additional filter unit. The selection of a specific optical filter or a specific observation mode can be made by choosing a suitably equipped exchangeable shaft.

The controller 66 is configured to detect a coupled exchangeable shaft 312, 314. This can be done software-based, mechanically and/or through sensor detection. Depending on the detected exchangeable shaft 312, 314, the controller 66 can then determine in which operating state or in which observation mode the imaging unit 14 should be operated. The control unit 66 is also configured to adjust an illumination mode of the illumination unit 18. The control unit 66 is thus configured to adjust an illumination mode of the illumination unit 18 on the basis of the observation mode defined by a currently coupled exchangeable shaft 312, 314.

In the present case, the exchangeable shafts 312, 314 and the imaging device 10 are part of a medical imaging system 316. The medical imaging system 316 allows a user to select a suitable exchangeable shaft 312, 314, couple it to the base unit 310, and thereby define a mode for the entire imaging device 10. By simply changing the exchangeable shaft 312, 314, it is thus achieved that the illumination device 18 is automatically adapted to the image acquisition mode to be used.

FIG. 7 shows a schematic perspective representation of another embodiment of an imaging device 10′. The reference signs of this embodiment are provided with inverted commas for differentiation purposes. In this embodiment, the imaging device 10′ is designed as an exoscopic imaging device. It comprises an illumination device 12′ and an imaging unit 14′. Its basic functionality corresponds to that described above, but in this embodiment the imaging unit 14′ is configured as an exoscope.

Aspects of the above description can also be summarized or described as follows. FIG. 8 shows a schematic flow chart of a method for generating illumination light for an imaging unit 14 by means of an illumination device 12. The sequence of the method is also clear from the above explanations. The illumination device 12 comprises an optical interface 16 for optically connecting an imaging unit 14, and an illumination unit 18 configured to provide illumination light to the optical interface 16, wherein the illumination unit 18 comprises a plurality of luminous elements 20, 22, 24, 26, 28 which are optionally activatable independently of one another and which are configured to emit light according to different emission spectra in order to provide the illumination light.

The method comprises a step S11 of at least temporarily activating a first group of the luminous elements 20, 22, 24, 26, 28 in order to provide illumination light for multispectral imaging. The method further comprises a step S12 of at least temporarily activating a second group of the luminous elements 20, 22, 24, 26, 28 in order to provide illumination light for fluorescence imaging. One of the luminous elements 20, 22, 24, 26, 28 is activated at least temporarily both when the first group of luminous elements 20, 22, 24, 26, 28 is activated at least temporarily and when the second group of luminous elements 20, 22, 24, 26, 28 is activated at least temporarily.

FIG. 9 shows a schematic flow chart of a method for operating an imaging device 10. The sequence of the method is also clear from the above explanations. An imaging device 10 having an imaging unit 14 is provided in a step S21. Illumination light is provided to the imaging unit 14 in a step S22. The provision of illumination light to the imaging unit 14 is carried out according to a method as described with reference to FIG. 8.

FIG. 10 shows a schematic flow chart of a method for operating an imaging device 10. The sequence of the method is also clear from the above explanations. The method comprises a step S31 of providing an illumination device 12 for providing illumination light for an imaging unit 14. The imaging unit 14 comprises an optical interface 16 for optically connecting an imaging unit 14, and an illumination unit 18 that is designed to provide illumination light to the optical interface 16. The illumination unit 18 is multimodal and can be operated in a plurality of different illumination modes. The method further comprises a step S32 of providing an imaging unit 14 that can be connected to the optical interface 16 of the illumination device 12. The method moreover comprises a step S33 of automatically matching an operating state of the imaging unit 14 and an illumination mode of the illumination unit 18.

The devices, methods and systems described in general above and/or the devices, methods and systems described above using exemplary embodiments relate in particular to the following main aspects I, Il and Ill, the aspects of which are each numbered with Arabic numerals below and which can also be combined with one another, i.e., in particular main aspect I with main aspect II, main aspect I with main aspect III, main aspect Il with main aspect III and main aspect I with main aspect Il and main aspect Ill as well as their respective associated aspects:

### Main Aspect I

I-1. An illumination device (12), in particular for providing illumination light for an imaging unit (14) such as an endoscope, exoscope and/or microscope, comprising:
   an optical interface (16) for optically connecting an imaging unit (14); and
   an illumination unit (18) that is designed to provide illumination light to the optical interface (16),
      wherein the illumination unit (18) is multimodal and comprises a plurality of luminous elements (20, 22, 24, 26, 28) which are optionally activatable independently of one another and which are configured to emit light according to different emission spectra in order to provide the illumination light,
      wherein the illumination unit (18) may be operable in at least one multispectral mode in which a first group of the luminous elements (20, 22, 24, 26, 28) is at least temporarily activated and in which the illumination unit (18) provides illumination light for multispectral imaging;
      wherein the illumination unit (18) is operable in at least one fluorescence mode in which a second group of the luminous elements (20, 22, 24, 26, 28) is at least temporarily activated and in which the illumination unit (18) provides illumination light for fluorescence imaging; and
      wherein the luminous elements (20, 22, 24, 26, 28) comprise at least one luminous element (20) which is included in both the first group and the second group.
I-2. The illumination device (12) according to aspect I-1 or 1-2,
   wherein at least one luminous element (20) contained in both the first group and the second group emits light in the red spectral range, in particular in a spectral range between 600 nm and 680 nm.
I-3. The illumination device (12) according to aspect I-1,
   wherein the illumination unit (18) is operable in at least one white light mode in which the illumination unit (18) provides illumination light for white light imaging.
I-4. The illumination device (12) according to aspect I-3,
   wherein in the white light mode a third group of the luminous elements (20, 22, 24, 26, 28) is activated at least temporarily to provide the illumination light for white light imaging,
   and wherein the luminous elements (20, 22, 24, 26, 28) comprise at least one luminous element (20, 22, 24) which is contained both in the first group and/or second group and in the third group.
I-5. The illumination device (12) according to aspect I-4,
   wherein at least one luminous element (20) contained both in the first group and/or second group and in the third group emits light in the red spectral range, in particular in a spectral range between 600 nm and 680 nm.
I-6. The illumination device (12) according to aspect I-4 or I-5,
   wherein at least one luminous element (24) contained both in the first group and/or second group and in the third group emits light in the blue spectral range, in particular in a spectral range between 440 and 480 nm.
I-7. The illumination device (12) according to any of the preceding aspects of main aspect I,
   wherein the luminous elements (20, 22, 24, 26, 28) comprise at least one luminous element (22) which emits light in a spectral range between 750 and 790 nm and/or
   wherein the luminous elements (20, 22, 24, 26, 28) comprise at least one luminous element (28) which emits light in a spectral range between 920 and 960 nm.
I-8. The illumination device (12) according to any of the preceding aspects of main aspect I,
   wherein the illumination unit (18) comprises at least one crossed beam splitter (30, 32), by means of which light can be deflected from opposite input sides (34, 36, 38, 40) to an output side (42, 44), and wherein at least one of the luminous elements (20, 22, 24, 26, 28) is arranged in each case on the opposite input sides (34, 36, 38, 40) of the crossed beam splitter (30, 32).
I-9. The illumination device (12) according to any of the preceding aspects of main aspect I,
   wherein the luminous elements (20, 22, 24, 26, 28) comprise at least four narrowband emitting single-color luminous elements, each with different spectral ranges, and at least one broadband emitting white light luminous element.
I-10. The illumination device (12) according to any of the preceding aspects of main aspect I,
   wherein the illumination unit (18) is operable in at least one hyperspectral mode in which a plurality of luminous elements (20, 22, 24, 26, 28) is activated, the emission spectra of which together cover at least a spectral range from 450 nm to 850 nm, and in which the illumination unit (18) provides illumination light for hyperspectral imaging.
I-11. An imaging device (10) comprising:
   an illumination device (12) according to any of the preceding aspects of main aspect I; and
   an imaging unit (14) connectable to the optical interface (16) of the illumination device (12).
I-12. The imaging device (10) according to aspect I-11,
   wherein the imaging unit (14) comprises a filter unit (46) with optical filters (48, 50, 52) that can be switched at least between a multispectral mode and a fluorescence mode.
I-13. A method for generating illumination light for an imaging unit (14) by means of an illumination device (12), in particular by means of an illumination device (12) according to any of aspects I-1 to I-10,
   wherein the illumination device (12) comprises an optical interface (16) for optically connecting an imaging unit (14), and an illumination unit (18) configured to provide illumination light to the optical interface (16), wherein the illumination unit (18) comprises a plurality of luminous elements (20, 22, 24, 26, 28) which are optionally activatable independently of one another and which are configured to emit light according to different emission spectra in order to provide the illumination light,
   the method comprising:
      at least temporarily activating a first group of the luminous elements (20, 22, 24, 26, 28) in order to provide illumination light for multispectral imaging; and
      at least temporarily activating a second group of the luminous elements (20, 22, 24, 26, 28) in order to provide illumination light for fluorescence imaging; and
      wherein at least one of the luminous elements (20, 22, 24, 26, 28) is activated at least temporarily both when the first group of luminous elements (20, 22, 24, 26, 28) is activated at least temporarily and when the second group of luminous elements (20, 22, 24, 26, 28) is activated at least temporarily.
I-14. A method for operating an imaging device (10), in particular according to any of aspects I-11 or I-12,
   wherein the imaging device (10) comprises an imaging unit (14),
   wherein according to a method according to aspect I-13, illumination light is provided to the imaging unit (14).

### Main Aspect II

II-1. A medical imaging device (10), in particular an endoscopic and/or exoscopic and/or microscopic imaging device, comprising:
   an illumination device (12) for providing illumination light for an imaging unit (14), comprising:
   an optical interface (16) for optically connecting an imaging unit (14); and
   an illumination unit (18) that is designed to provide illumination light to the optical interface (16), wherein the illumination unit (18) is multimodal and operable in a plurality of different illumination modes; and
   an imaging unit (14) connectable to the optical interface (16) of the illumination device (12); and
   a controller (66) which is configured to automatically match an operating state of the imaging unit (14) and an illumination mode of the illumination unit (18) to one another.
II-2. The medical imaging device (10) according to aspect II-1,
   wherein the illumination mode of the illumination unit (18) and/or the operating state of the imaging unit (14) can be specified by at least one user action, and wherein the controller (66) is configured to automatically match, in response to the user action, the operating state of the imaging unit (14) and the illumination mode of the illumination unit (18) to one another.
II-3. The medical imaging device (10) according to aspect II-1 or II-2,
   wherein the imaging unit (14) comprises a camera unit (68), and wherein the controller (66) is configured to adjust an illumination mode of the illumination unit (18) on the basis of an operating state of the camera unit (68).
II-4. The medical imaging device (10) according to aspect II-3,
   wherein the camera unit (68) comprises a plurality of optical filters (48, 50, 52) that can optionally be introduced into an observation beam path (70) of the camera unit (68) and that define different observation modes that can be selected by a user, and wherein the controller (66) is configured to adjust the illumination mode on the basis of a selected observation mode.
II-5. The medical imaging device (10) according to aspect II-4,
   wherein the optical filters (48, 50, 52) can be manually introduced into the observation beam path (70) by the user, wherein the camera unit (68) comprises at least one filter sensor (72) which is configured to automatically detect an optical filter (48, 50, 52) currently introduced into the observation beam path (70) and to generate a sensor signal which contains information relating to the detected optical filter (48, 50, 52), and wherein the controller (66) is configured to detect the observation mode of the camera unit (68) in accordance with the sensor signal.
II-6. The medical imaging device (10) according to any of aspects II-4 to II-5,
   wherein the camera unit (68) comprises an automated filter unit (210) which is configured to automatically introduce at least one of the optical filters (48, 50, 52) into the observation beam path (70) in accordance with an observation mode specified by a user.
II-7. The medical imaging device (10) according to any of aspects II-3 to II-6,
   wherein the imaging unit (14) comprises a distal shaft (76), wherein the camera unit (68) is a proximal camera unit, and wherein the shaft (76) is optically coupled to the camera unit (68).
II-8. The medical imaging device (10) according to any of the preceding aspects of main aspect II,
   wherein the illumination unit (18) comprises a plurality of luminous elements (20, 22, 24, 26, 28) which are optionally activatable independently of one another and which are configured to emit light according to different emission spectra in order to provide the illumination light, and
   wherein the illumination unit (18) is operable in at least one multispectral mode in which the illumination unit (18) provides illumination light for multispectral imaging, and/or wherein the illumination unit (18) is operable in at least one fluorescence mode in which the illumination unit (18) provides illumination light for fluorescence imaging, and/or wherein the illumination unit (18) is operable in at least one white light mode in which the illumination unit (18) provides illumination light for white light imaging.
II-9. The medical imaging device (10) according to any of the preceding aspects of main aspect II,
   wherein the imaging unit (14) comprises a broadband transmitting optics (77) that can be used uniformly in the different illumination modes.
II-10. The medical imaging device (10) according to any of the preceding aspects of main aspect II,
   wherein the imaging unit (14) comprises a proximal base unit (310) to which different exchangeable shafts (312, 314) designed for different observation modes can optionally be optically and electronically coupled, and wherein the controller (66) is configured to adjust an illumination mode of the illumination unit (18) on the basis of the observation mode defined by a currently coupled exchangeable shaft (312, 314).
II-11. A medical imaging system (316), comprising:
   a medical imaging device (10) according to aspect II-10; and
   at least two different exchangeable shafts (312, 314) which can be optionally connected to the base unit (310) of the imaging unit (14).
II-12. The medical imaging system (316) according to aspect II-11,
   wherein the exchangeable shafts (312, 314) each comprise an integrated camera (318, 320) and/or integrated optical filters (322, 324).
II-13. A method for operating a medical imaging device (10), in particular according to any of aspects II-1 to II-10,
   wherein the imaging device (10) comprises:
      an illumination device (12) for providing illumination light for an imaging unit (14), comprising:
      an optical interface (16) for optically connecting an imaging unit (14); and
      an illumination unit (18) that is designed to provide illumination light to the optical interface (16), wherein the illumination unit (18) is multimodal and operable in a plurality of different illumination modes; and
   an imaging unit (14) connectable to the optical interface (16) of the illumination device (12);
   and wherein the method comprises:
   automatically matching an operating state of the imaging unit (14) and an illumination mode of the illumination unit (18).

### Main Aspect III

III-1. An illumination device (12), in particular for providing illumination light for an imaging unit (14) such as an endoscope, exoscope and/or microscope, comprising:
   an optical interface (16) for optically connecting an imaging unit (14); and
   an illumination unit (18) that is designed to provide illumination light to the optical interface (16),
      wherein the illumination unit (18) comprises a plurality of luminous elements (20, 22, 24, 26, 28) which are optionally activatable independently of one another and which are configured to emit light according to different emission spectra in order to provide the illumination light,
      wherein the illumination unit (18) is operable in at least one multispectral mode in which a first group of the luminous elements (20, 22, 24, 26, 28) that comprises at least two of the luminous elements (20, 26) is at least temporarily activated and in which the illumination unit (18) provides illumination light for multispectral imaging;
      wherein the luminous elements (20, 26) of the first group are arranged such that light emitted by the luminous elements (20, 26) travels through a light path of at least substantially the same length from the relevant luminous element (20, 26) to the optical interface (16).
III-2. The illumination device (12) according to aspect III-1,
   wherein the illumination unit (18) defines a common optical path (54) into which emitted light from the luminous elements (20, 22, 24, 26, 28) can be coupled, wherein the luminous elements (20, 26) of the first group each have a light-emitting surface (56, 58), and wherein the light-emitting surfaces (56, 62) of the luminous elements (20, 26) of the first group are arranged equidistantly with respect to the common optical path (54).
III-3. The illumination device (12) according to aspect III-1 or III-2,
   wherein the illumination unit (18) comprises at least one crossed beam splitter (32), by means of which light can be deflected from opposite input sides (38, 40) to an output side (44), and wherein at least one of the luminous elements (20, 26) of the first group is arranged in each case on the opposite input sides (38, 40) of the crossed beam splitter (32).
III-4. The illumination device (12) according to aspect III-2 and III-3,
   wherein the crossed beam splitter (32) is arranged such as to couple light coming from the opposite input sides (38, 40) into the common optical path (54).
III-5. The illumination device (12) according to aspect III-3 or III-4,
   wherein the at least one beam splitter (32) comprises at least three input sides (38, 40, 41), two of which form the opposite input sides (38, 40) and a third of which is opposite the output side (44).
III-6. The illumination device (12) according to any of aspects III-3 to III-5,
   wherein the illumination unit (18) comprises at least two crossed beam splitters (30, 32) arranged optically one behind the other.
III-7. The illumination device (12) according to any of the preceding claims of main aspect III,
   wherein the illumination unit (18) is operable in at least one fluorescence mode in which a second group of the luminous elements (20, 22, 24, 26, 28) is at least temporarily activated and in which the illumination unit (18) provides illumination light for fluorescence imaging, and/or
   wherein the illumination unit (18) is operable in at least one white light mode in which the illumination unit (18) provides illumination light for white light imaging.
III-8. The illumination device (12) according to any of the preceding aspects of main aspect III,
   wherein the illumination unit (18) is operable in at least one hyperspectral mode in which a plurality of luminous elements (20, 22, 24, 26, 28) is activated, the emission spectra of which together cover at least a spectral range from 450 nm to 850 nm, and in which the illumination unit (18) provides illumination light for hyperspectral imaging.
III-9. An imaging device (10) comprising:
   an illumination device (12) according to any of the preceding aspects of main aspect III; and
   an imaging unit (14) connectable to the optical interface (16) of the illumination device (12).
III-10. The imaging device (10) according to aspect III-9,
   wherein the imaging unit (14) comprises a filter unit (46) with optical filters (48, 50, 52) that can be switched at least between a multispectral mode and a fluorescence mode and/or a white light mode.

### LIST OF REFERENCE SIGNS

10 Imaging device
12 Illumination device
14 Imaging unit
16 Optical interface
18 Illumination unit
20 Luminous element
22 Luminous element
24 Luminous element
26 Luminous element
28 Luminous element
30 Beam splitter
32 Beam splitter
34 Input side
36 Input side
37 Input side
38 Input side
40 Input side
41 Input side
42 Output side
44 Output side
46 Filter unit
48 Filter
50 Filter
52 Filter
54 Optical path
56 Light-emitting surface
58 Light-emitting surface
60 Light-emitting surface
62 Light-emitting surface
64 Light-emitting surface
66 Controller
68 Camera unit
70 Observation beam path
72 Filter sensor
74 Display unit
76 Shaft
77 Optics
78 Lens
80 Lens
82 Lens
84 Lens
86 Lens
88 Lens
90 Beam splitter element
92 Beam splitter element
94 Beam splitter element
96 Beam splitter element
98 Transmission spectrum
100 Transmission spectrum
102 Transmission spectrum
104 Transmission spectrum
106 Light guide
108 Imaging sensor system
110 White light sensor
112 Near-IR sensor
114 Light path
116 Distal portion
210 Filter unit
212 Filter drive
214 User interface
310 Base unit
312 Exchangeable shaft
314 Exchangeable shaft
316 Imaging system
318 Camera
320 Camera
322 Filter
324 Filter

1-37 (canceled)

38. An illumination device for providing illumination light for an imaging unit such as
an endoscope, exoscope and/or microscope, comprising:
an optical interface for optically connecting an imaging unit; and
an illumination unit configured to provide illumination light to the optical interface,
wherein the illumination unit is multimodal and comprises a plurality of luminous elements that are optionally activatable independently of one another and that are configured to emit light according to different emission spectra in order to provide the illumination light;
wherein the illumination unit may be operable in at least one multispectral mode in which a first group of the luminous elements is at least temporarily activated, and in which the illumination unit provides illumination light for multispectral imaging;
wherein the illumination unit is operable in at least one fluorescence mode in which a second group of the luminous elements is at least temporarily activated and in which the illumination unit provides illumination light for fluorescence imaging; and
wherein the luminous elements comprise at least one luminous element which is included in both the first group and the second group.

39. The illumination device of claim 38, wherein at least one luminous element contained in both the first group and the second group emits light in the red spectral range.

40. The illumination device of claim 39, wherein the red spectral range wavelengths between 600 nm and 680 nm

41. The illumination device of claim 38, wherein the illumination unit is operable in at least one white light mode in which the illumination unit provides illumination light for white light imaging.

42. The illumination device of claim 41, wherein, in the white light mode, a third group of the luminous elements is activated at least temporarily to provide the illumination light for white light imaging, and wherein the luminous elements comprise at least one luminous element which is contained both in the first group and/or second group and in the third group.

43. The illumination device of claim 42, wherein at least one luminous element contained both in the first group and/or second group and in the third group emits light in the red spectral range.

44. The illumination device of claim 42 wherein at least one luminous element contained both in the first group and/or second group and in the third group emits light in the blue spectral range, in particular in a spectral range between 440 and 480 nm.

45. The illumination device of claim 43, wherein at least one luminous element contained both in the first group and/or second group and in the third group emits light in the blue spectral range, in particular in a spectral range between 440 and 480 nm.

46. The illumination device of claim 1, wherein the luminous elements comprise at least one luminous element which emits light in a spectral range between 750 and 790 nm and/or wherein the luminous elements comprise at least one luminous element which emits light in a spectral range between 920 and 960 nm.

47. The illumination device of claim 1, wherein the illumination unit comprises at least one crossed beam splitter, by means of which light can be deflected from opposite input sides to an output side, and wherein at least one of the luminous elements is arranged in each case on the opposite input sides of the crossed beam splitter.

48. The illumination device of claim 1, wherein the luminous elements comprise at least four narrowband emitting single-color luminous elements, each with different spectral ranges, and at least one broadband emitting white light luminous element.

49. The illumination device of claim 47, wherein the luminous elements comprise at least four narrowband emitting single-color luminous elements, each with different spectral ranges, and at least one broadband emitting white light luminous element.

50. The illumination device of claim 1, wherein the illumination unit is operable in at least one hyperspectral mode in which a plurality of luminous elements is activated, the emission spectra of which together cover at least a spectral range from 450 nm to 850 nm, and in which the illumination unit provides illumination light for hyperspectral imaging.

51. The illumination device of claim 48, wherein the illumination unit (18) is operable in at least one hyperspectral mode in which a plurality of luminous elements is activated, the emission spectra of which together cover at least a spectral range from 450 nm to 850 nm, and in which the illumination unit provides illumination light for hyperspectral imaging.

52. An imaging device comprising:
an illumination device for providing illumination light for an imaging unit such as an endoscope, exoscope and/or microscope, comprising an optical interface for optically connecting an imaging unit, and an illumination unit configured to provide illumination light to the optical interface, wherein the illumination unit is multimodal and comprises a plurality of luminous elements that are optionally activatable independently of one another and that are configured to emit light according to different emission spectra in order to provide the illumination light, wherein the illumination unit may be operable in at least one multispectral mode in which a first group of the luminous elements is at least temporarily activated, and in which the illumination unit provides illumination light for multispectral imaging, wherein the illumination unit is operable in at least one fluorescence mode in which a second group of the luminous elements is at least temporarily activated and in which the illumination unit provides illumination light for fluorescence imaging, and
wherein the luminous elements comprise at least one luminous element which is included in both the first group and the second group; and
an imaging unit connectable to the optical interface of the illumination device.

53. The imaging device of claim 52, wherein the imaging unit comprises a filter unit with optical filters that can be switched at least between a multispectral mode and a fluorescence mode.

54. A method for generating illumination light for an imaging unit, comprising the steps of
providing an illumination device, the illumination device comprising an optical interface configured to optically connect an imaging unit;
providing an illumination unit configured to provide an illumination light to an optical interface, wherein the illumination unit comprises a plurality of luminous elements which are optionally activatable independently of one another and which are configured to emit light according to different emission spectra in order to provide the illumination light;
at least temporarily activating a first group of the luminous elements in order to provide illumination light for multispectral imaging; and
at least temporarily activating a second group of the luminous elements in order to provide illumination light for fluorescence imaging,
wherein at least one of the luminous elements is activated at least temporarily both when the first group of luminous elements is activated at least temporarily and when the second group of luminous elements is activated at least temporarily.

55. The method for generating illumination light for an imaging unit of claim 54,
comprising the further step of operating an imaging device, the imaging device comprising the imaging unit, the illumination device providing, thereby, illumination light to the imaging unit.

56. The method for generating illumination light for an imaging unit of claim 54, comprising the further step of operating an imaging device, the imaging device comprising the imaging unit and a filter unit with optical filters configured to be switchable at least between a multispectral mode and a fluorescence mode, the illumination device providing, thereby, illumination light to the imaging unit.

57. The method for generating illumination light for an imaging unit of claim 56,
comprising the further step of switching between the multispectral mode and the fluorescence mode.
